(19) United States
(12) Patent Application Publication
(10) Pub. No.: US 2026/0263139 A1
(43) Pub. Date: Sep. 10, 2026

(54) DEVICE AND METHOD FOR TREATMENT OF PART OF A HUMAN BODY

(71) Applicant: BTL Medical Solutions a.s., Prague 1 (CZ)

(72) Inventors: Tomáš Schwarz, Praha (CZ); Pavel Mykytyn, Praha 5 (CZ)

(21) Appl. No.: 18/868,003

(22) PCT Filed: May 25, 2023
(86) PCT No.: PCT/IB2023/000330

§ 371 Date: Nov. 21, 2024

Related U.S. Application Data
(60) Provisional application No. 63/345,469, filed on May 25, 2022.

(51) Int. Cl. A61B 18/12; A61B 18/00; A61B 18/14

(52) U.S. Cl. CPC A61B 18/1206; A61B 18/14; A61B 2018/00458; A61B 2018/1273; A61B 2018/143; A61B 2018/1475

(57) ABSTRACT

A system for treatment of at least part of a human body comprises one or more generators, the one or more generators configured to generate radiofrequency signals and pulsed electric field (PEF) signals, one or more applicators electrically coupled to the one or more generators, the applicators configured to deliver radiofrequency field and PEF to at least part of the human body based on the radiofrequency signals and PEF signals from the one or more generators, and at least one control unit, in communication with the one or more generators and/or one or more applicators, the control unit configured to control the delivery of at least one of the radiofrequency field and the PEF to at least part of the human body. An applicator for treating at least part of a body and a method of treating at least part of a body are also disclosed.

### CROSS-REFERENCE TO RELATED APPLICATIONS

This application claims priority to US Provisional Patent Application No. 63/345,469, filed on May 25, 2022, incorporated herein by reference in its entirety.

### FIELD OF THE INVENTION

The invention relates to devices and methods of a treatment of at least part of a human body by application of a combination of radiofrequency energy and electric field into a tissue of at least part of a human body.

### BACKGROUND OF THE INVENTION

A human or an animal body is covered by a skin. A skin tissue on the surface of a human or animal body includes the epidermis, dermis and hypodermis. The epidermis is the outermost and the thinnest layer of the skin. The second layer called the dermis includes connective tissue, reticular fibers, hair follicle roots, lymphatic vessels, collagen tissue and nerves. The third layer of the skin, sometimes called the hypodermis, is the lowest layer of the skin and may contain a fat forming subcutaneous white adipose tissue. Body cavities include layers which may be more or less differentiated and may include for example a mucosal tissue. For example, tissue layers in the vaginal cavity include the epithelium, the lamina propria (having a similar composition to the dermis), muscles and adventitia.

Recently a so-called fractional treatment of a tissue, for example of a skin, adipose tissue or mucosal tissue has been introduced. Such a treatment uses an effect of a point-like, spatially delimited tissue damage to induce natural healing processes in the tissue. The spatially delimited areas of a damaged tissue are usually surrounded by an undamaged tissue. Such tissue damage is usually achieved by use of electromagnetic energy (for example lasers) or for example radiofrequency (RF) energy for example RF field, which may be delivered by superficial or invasive surface energy delivery elements, for example by flat or needle-like energy delivery elements placed on an applicator. An RF field may be delivered in a monopolar or a bipolar manner and the distances between particular energy delivery elements of the applicator may ensure spatially delimited (fractional) skin damage. During such a procedure the RF field causes a temperature increase in a tissue zone adjacent to the energy delivery elements above a certain limit (for example a coagulation limit) thus killing tissue cells (for example by necrosis, for example coagulative necrosis) and damaging or destroying (for example by coagulation) non-cellular structures in the zone. Such damaged tissue is then restored by a natural healing process especially driven from the cells from the undamaged areas outside the damaged zones.

The RF fractional treatment has some disadvantages. The undamaged cells outside the damaged zones, or at least some of them, may be already relatively old or may become senescent. Such old cells or senescent cells then may not be as effective in driving the healing process compared to young cells. It would then be advantageous to replace the old or senescent cells with new ones in the undamaged areas. The replacement of the old or senescent cells by killing them by heat caused by an RF field is however obviously not optimal, since the heat may cause necrosis of the cells and may damage non-cellular structures in the areas where it is not desirable, which may cause an even longer healing process. Hence, the current methods and devices need to be improved to provide new or better treatment results.

### SUMMARY OF THE INVENTION

Disclosed herein are devices and methods of treatment, in particular devices and methods for a treatment of at least part of a human body by radiofrequency (RF) energy, for example by RF field, and by an electric field, for example by a pulsed electric field (PEF).

The surprising positive synergic effect of a combination of application of the radiofrequency field and the pulsed electric field (PEF) may provide advantages over current methods using only RF energy by addressing and solving the above mentioned problems and by enhancing quality, efficacy and reliability of the methods.

### BRIEF DESCRIPTION OF THE DRAWINGS

An exemplary aspect of the present disclosure is illustrated by way of example in the accompanying drawings in which like reference numbers indicate the same or similar elements and in which:

FIG. 1 is a block diagram of an exemplary system.

FIG. 2 is an overview of an exemplary applicator.

FIG. 3 shows an example of a distal end of the applicator with superficial energy delivery elements.

FIG. 4 shows another example of a distal end of the applicator, this time with invasive energy delivery elements.

FIG. 5 shows an example of the applicator with invasive and superficial energy delivery element.

FIG. 6 shows a detail of exemplary invasive energy delivery elements.

FIG. 7 shows a part of an exemplary RF treatment protocol.

FIG. 8 shows a part of an exemplary PEF treatment protocol.

FIG. 9 is an example of a combination of RF pulses and PEF pulses in one protocol.

FIG. 10 shows an example of an exemplary PEF generator.

### DETAILED DESCRIPTION

The combined application of RF energy, for example of an RF field and PEF may bring advantages to a process of treating at least a part of a human body, for example a treatment of a skin, a mucosal tissue or for example an adipose tissue. As already stated above, an RF therapy, for example so called fractional RF therapy, including a delivery of an RF field, for example at least one AC current pulse (RF pulse) into a tissue, destroys a tissue by heating to a high temperature, for example by heating to 43-120° C. or by 50-95° C. or by 60-85° C. The high temperature may cause for example a hemocoagulation of cells and non-cellular structures like collagen and/or elastin. A therapy using electric fields, for example pulsed electric fields (PEF), including a delivery of for example at least one direct current (DC) electric pulse (PEF pulse), may affect mainly cells while avoiding damage to non-cellular structures. The cells may be destroyed primarily not by high temperatures (the tissue temperature during this therapy may not exceed 42° C.), but by a process called electroporation of cell membranes. The tissue damage by the RF therapy may be spatially delimited (fractional), because the application of the RF field may heat and thus damage cells and non-cellular structures in the area (zone) of the tissue in a close proximity to the energy delivery element while the tissue damage done by PEF therapy may be more continuous, because the electric field may be more homogenous compared to the RF field. The combination of the application of the two therapies (RF and PEF field) may result in at least one first area in a treated tissue where cells and non-cellular tissue are destroyed by high temperature (cells may die for example by necrosis) and with a second area, where cells may be destroyed by an electroporation of cell membranes and may die for example by apoptosis. The first area may spread in close proximity to and/or around the at least one energy delivery element. The second area may intersect the first area or may be separated from the first area and may spread beyond the area (zone) of the tissue damaged by the RF.

The cells destroyed by an electroporation of cell membranes, for example the cells in the second area of the treated tissue, among others, for example old cells or senescent cells, may be quickly replaced by new cells and these new cells then may drive a healing process in the treated tissue including the first area, for example by production of new non-cellular structures in the treated tissue including the first area.

Although electroporation of cell membranes is described as the primary trigger of death of the cells after the application of PEF, the actual cell death may alternatively be caused for example by an electrical breakdown of the membrane of mitochondria or nucleus; by a tearing of individual cells (or groups of cells) of the tissue, either directly by electric fields or by mechanical damage; by cell myolysis; by wrinkling cells either directly under the influence of the electric field or by mechanical damage; by irreversible damage to the calcium cycle; by calcium overload for example by non-physiological function of one or more calcium channels; or by formation of reactive oxygen species (ROS) and subsequent oxidation of membrane phospholipids by electric field. Further the cells may be killed by a combination of elevated temperature, and PEF, wherein the temperature is not high enough to kill the cells on its own.

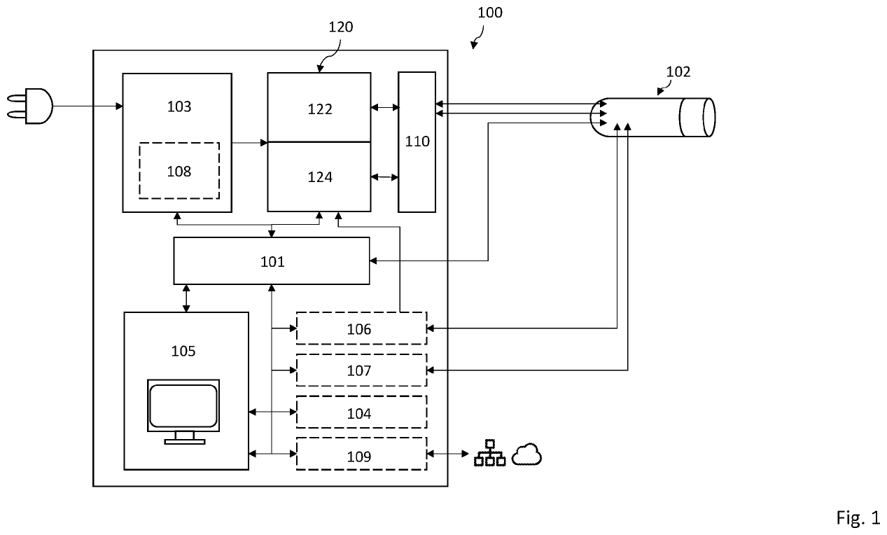

The system (100) according to this invention as shown in FIG. 1 may include one or more generators (120) suitable for providing radiofrequency (RF) signals adapted for creation of an RF field (RF signals)—RF generator (122) and/or for providing electrical signals adapted for creation of a pulsed electric field (PEF signals)—PEF generator (124). The system (100) may further include at least one control unit (101), at least one applicator (102), at least one power supply unit (103), a data storage unit (104), at least one switching unit (110) and at least one user interface unit (105). It may also include other parts, for example a cooling unit (106) suitable for cooling of a part of the system (100) or of at least part of a human body or a fluid delivery unit (107) suitable for delivery of a fluid adjacent to at least part of a human body.

The at least one control unit (101) may be communicatively coupled to one or more of the generators (120), with the switching unit (110), and/or with the at least one applicator (102) and may, for example control the providing of at least one of the RF signals and the PEF signals from the one or more generators into the at least one energy delivery element, and hence control the delivery of at least one of RF field and PEF into at least part of a human body according to a treatment protocol. The at least one control unit may be further communicatively coupled to the user interface unit (105) from which it may receive instructions. It may also be communicatively coupled to other parts of the system (100), for example with the at least one power supply unit (103), cooling unit (106) and/or fluid delivery unit (107), or for example with at least one sensor included in the system (100). The control unit (101) may include for example a microprocessor and/or a memory.

The user interface unit (105) may be configured for communication with a human, for example with an operator of the system (100) and may serve as a communication interface of the system (100). It may be configured to receive instructions from and/or to present information to the human operator. The communication may for example include graphical communication via for example at least one display unit, a sound communication via for example at least one microphone and/or at least one speaker, or a haptic communication via for example at least one touch display, keyboard, mouse, knob, joystick or other similar device suitable for haptic communication.

The at least one power supply unit (103) may include for example an energy source, for example a connection to an electricity network or at least one battery (108). It may also include an AC/DC adapter and/or may be in an electrical coupling with one or more generators (120) and with other parts of the system (100), particularly with the parts which require electrical energy, for example with the control unit (101), user interface unit (105), switching unit (110), and/or with at least one applicator (102), and may supply them with electrical energy.

The whole system (100) may be included in one single device, or it may be divided into several separate devices. It may also be connected to a network, for example the internet or a data server via network connection (109), for example via Ethernet or Wi-Fi. It may include data storage for example comprising a database, however the database may be stored externally, for example in the data server and may be reachable via connection to a network. The database may for example include treatment protocols, a therapy database, a parameter (for example a therapy parameter) database and/or a patient database.

The system (100) may include one or more generators (120) suitable for providing radiofrequency (RF) signals adapted for creation of an RF field (RF signals)—RF generator (122) and/or for providing electrical signals adapted for creation of a pulsed electric field (PEF signals)—PEF generator (124). The system (100) may include a single generator (120), configured to provide both the RF and the PEF signals or it may include more than one generator (120), for example at least one RF generator (122) and at least one PEF generator (124). The one or more generators (120) may be for example electrically coupled to the at least one applicator (102), for example with at least one energy delivery element, and with the power supply unit (103). It may be communicatively coupled to other parts of the system (100), for example with the control unit (101), user interface unit (105), applicator (102) etc. The one or more generators (120) may also be coupled to the cooling unit (106) (for example by a fluid connection).

The RF generator provides RF AC signals for at least one energy delivery element, which generates an RF field based on the RF signals from the RF generator and applies it (delivers it) to at least part of a human body to cause heating in a target tissue. The RF generator may have the following exemplary properties-acceptable loads may be 10Ω-15000Ω, rated load 50Ω-5000Ω, a rated RF power may be from 1 W to 500 W.

The PEF generator provides electric direct current (DC) signals for at least one energy delivery element. The at least one delivery element generates an electric field, for example PEF, and applies it (delivers it) to at least part of a human body to cause electroporation of cell membranes in a treated tissue. The PEF generator may have a power input of 0.01 W to 600 W, and power output of from 0.01 to 500 W. The PEF generator may include a Marx-type generator and/or at least one transformer. A pulse generator may allow settings for example of a pulse width, an amplitude, a shape of the PEF pulse and/or a number of pulses during activation. The pulse generator or other part of the device may be able to measure electrical waveforms qualities, power, impedance, electric current, voltage, temperature and/or other variables.

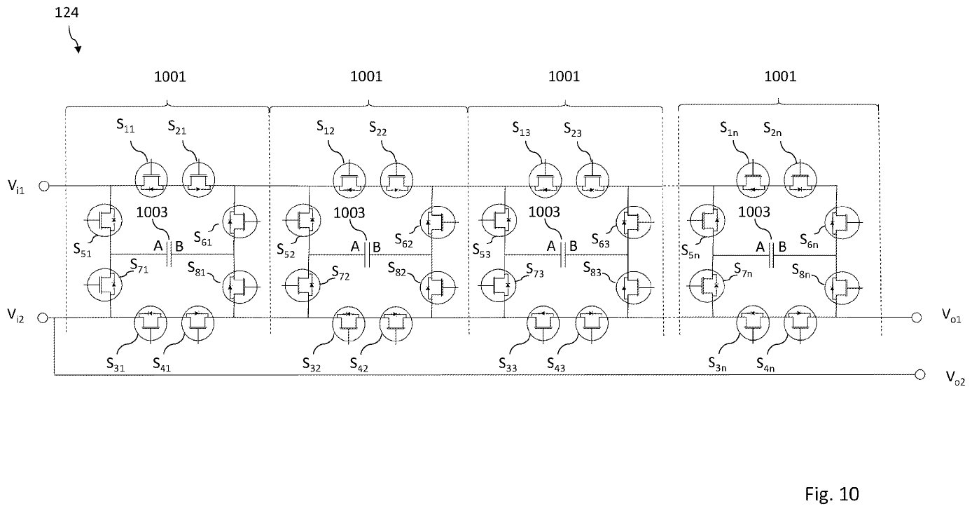

The PEF generator may be for example a Marx type generator, for example with semiconductor switches. The generator may include a plurality of capacitors and plurality of switches. The capacitors are charged in parallel configuration and discharged in series configuration. The generator may have monophasic configuration for generation of either positive or negative pulses only or biphasic configuration for generation of positive and negative pulses alternatively. FIG. 10 shows an example of an exemplary PEF generator (124). The PEF generator (124) is based on a Marx type generator with semiconductor switches. This exemplary design includes plurality of a common integrated pulse voltage multiplier stages (1001) (generator stage), wherein each voltage multiplier stage (1001) includes a single storage element (1003) (for example a capacitor) for delivering monophasic (both positive and negative) and biphasic pulses and and multiple semiconductor switches (S₁ₓ-S₈ₓ). The exemplary PEF generator (124) may have a more compact design compared to two unipolar generators. The PEF generator (124) may have one input (Vᵢ₁) wherein the Vᵢ₁ is a DC voltage input into the generator and one output (Vₒ₁), where the Voi is a high voltage output from the generator. The PEF generator (124) may have two ground terminals (Vᵢ₂, Vₒ₂) as well. The number of voltage multiplier stages (1001) included in the PEF generator (124) depends on a pulse voltage (amplitude) needed and maybe for example from 2 to 30 or from 3 to 10. The exemplary PEF generator (124) uses all of the storage elements (1003), hence all of the voltage multiplier stages (1001) included in the PEF generator (124), to produce the monophasic (both positive and negative) and biphasic pulse.

To charge the PEF generator (124) for a positive pulse, the switches S₅ₓ and S₈ₓ are non-conducting, while all other switches in all voltage multiplier stages (1001) are conducting. During the positive charging phase, the output (Vₒ₁) from the PEF generator (124) is disconnected (for example to disconnect a coupling of an applicator or energy delivery elements from the PEF generator (124)). This configuration allows the storage elements (1003) to be charged in parallel mode from positive side, which is in this case the side B of the storage elements (1003) to negative side, which is this time side A of the storage elements (1003). To discharge the positive pulse from the PEF generator (124) into the output (Vₒ₁), the switches S₇ₓ and S₈ₓ are conducting while all other switches in all voltage multiplier stages (1001) of the PEF generator (124) are non-conducting. In this configuration the storage elements (1003) are serially discharged in opposite directions compared to the positive charging phase from side A (this time positive) to side B (this time negative).

To charge the PEF generator (124) for a negative pulse, the switches S₆ₓ and S₇ₓ are non-conducting. The output (Vₒ₁) is again disconnected during the negative charging phase (possibly to disconnect a coupling of an applicator or energy delivery elements from the PEF generator (124)). All other switches in all voltage multiplier stages (1001) are conducting.

The storage elements (1003) are again charged in parallel mode, but in direction from side A, which is positive this time to side B, which is negative. To discharge the negative pulse from the generator serially, the switches S₇ₓ and S₈ₓ are conducting while all other switches in all voltage multiplier stages (1001) of the PEF generator (124) are non-conducting. The storage elements (1003) are serially discharged again in opposite direction compared to the negative charging phase from side B (this time positive) to side A (this time negative).

The switches in the generator may be for example MOSFET (Metal Oxide Semiconductor Field Effect Transistor) semiconductor switches. This type of switch is very fast which allows the PEF generator (124) to produce very short DC pulses with pulse width in length of nanoseconds, for example from 2 ns. However, the semiconductor switches could be replaced with switches of another type, for example a relay. In case the switches S₁ₓ, S₂ₓ and S₃ₓ, S₄ₓ are replaced with switches of another type for example a type not including a diode, for example relay, the number of the switches may be reduced in a way that the pair S₁ₓ, S₂ₓ may be replaced by only one switch within one voltage multiplier stage (1001) and the pair S₃ₓ, S₄ₓ may be replaced with another one switch within one voltage multiplier stage (1001).

The switching unit may be coupled to the at least one generator and to the at least one applicator. It may be configured to switch between delivery of the RF signals and the PEF signals provided by the at least one generator to the at least one energy delivery element. The switching unit may include at least one switch or relay.

The system may further include at least one applicator (102). The applicator may be adapted for engagement with at least part of a human body. It may be further adapted for delivery of RF field and/or PEF into at least part of a human body. The applicator may be further adapted to be operated (manipulated) manually by a human, for example by an operator or it may be adapted to be operated without need of manual manipulation, for example it may be operated automatically. It may also be fixed. It may further include at least one means for an attachment to at least part of a human body, for example a strap, clamp, cuff, clasps, clip, adhesive surface, suction device etc. The applicator may be electrically coupled to or include the one or more generators and/or the one or more switching units. It may be further electrically coupled to the power supply unit or it may include its own energy source, for example at least one battery. It may be communicatively coupled to or include other parts of the system for example a control unit, user interface unit, data storage, network connection, sensor, etc. Other couplings of the applicator to the system are possible, for example a fluid coupling for example to the cooling unit, fluid delivery unit or the cooling unit and/or the fluid delivery unit may be part of the applicator. The applicator may further include for example one or more handles. It may also include at least one part suitable for engagement with at least a part of a human body, for example with a skin, hypodermis, dermis, adipose tissue or mucosal tissue. The at least one part suitable for engagement with at least part of a human body may be completely non-invasive (for example adapted to engage the at least part of a human body by contacting the surface only) or may include invasive portions or elements (for example adapted to be inserted into at least a part of the human body tissue for example to the skin, hypodermis, dermis, adipose tissue or mucosal tissue). The system may include one applicator for example suitable for delivery of RF field and/or PEF, or it may include more applicators suitable for delivery of RF field and/or PEF.

The applicator may include at least one energy delivery element included at the part suitable for engagement with at least a part of a human body. The energy delivery element may be for example at least one electrode, may include at least one electrode, may be part of at least one electrode or it may be coupled to at least one electrode. The energy delivery element may be configured to receive signals from the RF generator or the PEF generator and generate the RF field and/or the electric field, particularly PEF based on that signals, and to apply (deliver) the RF field and/or the PEF into at least part of a human body, to cause heating in case of the RF field and to cause electroporation of cell membranes in case of the PEF. The energy delivery element may be configured to deliver the AC RF field into at least part of a human body continuously or in a pulsed manner, and the DC electric field in a continuous manner or in a pulsed manner in the form of a PEF. The energy delivery element may be superficial, for example adapted to engage at least part of a human body only by touching the surface. It may for example take the form of a surface electrode or a superficial pin. The superficial energy delivery element does not penetrate into the tissue before an application of at least one of the RF field and the PEF. In another aspect, the energy delivery element may be invasive, for example adapted to be inserted into at least part of a human body tissue for example to a skin, hypodermis, dermis, adipose tissue or mucosal tissue. It may for example take the form of a needle or a blade or any type of a sharp protrusion suitable for penetration into the tissue. The invasive energy delivery element is adapted to penetrate into a tissue before an application of at least one of the RF field and the PEF.

The superficial energy delivery element may be in some cases used for an invasive application of at least one of the RF field and the PEF. For example, a superficial pin may be within a therapy, or an application of the at least one applicator first brought into contact with a tissue without any penetration, however during or after a delivery of at least one of the RF field and the PEF it may, at least partially, penetrate into the tissue.

The at least one applicator may include one type of energy delivery element or it may include more types of them. For example one or more of the energy delivery elements included on the applicator may be invasive. In another example, the one or more of the energy delivery elements included on the applicator may be superficial. A combination of at least one superficial energy delivery element and at least one invasive energy delivery element on one applicator is also possible.

In another aspect the one or more energy delivery elements included on the applicator may be adapted to deliver both the RF field and the PEF or the applicator may comprise more than one energy delivery element each adapted for delivery of at least one of the RF field and the PEF. For example, the applicator may include one or more energy delivery elements adapted for delivery of the RF field and another one or more energy delivery elements adapted for a delivery of the PEF. The applicator may also include at least one energy delivery element configured to deliver the RF field and the PEF and at least one other energy delivery element configured for delivery of either an RF field or PEF, or the applicator may include a combination of all of the described energy delivery elements. For example, the applicator may include at least one invasive energy delivery element adapted for delivery of the RF field and at least one superficial energy delivery element adapted for delivery of the PEF or vice versa.

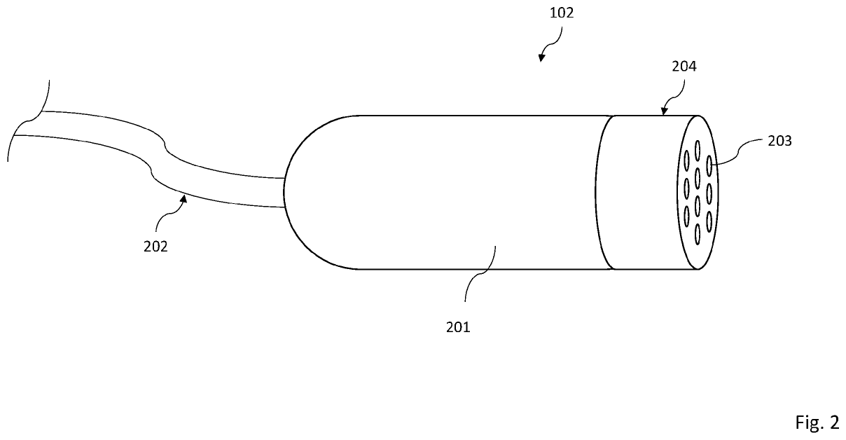

An example of the applicator (102) adapted for engagement with at least part of a human body, for example with a skin, may be seen in FIG. 2. The exemplary applicator may include a proximal part (201), which may be for example suitable for engagement with a hand of an operator. The proximal part (201) may be a handle, or may include a handle, and may include at least one connection (202), for example an electrical connection to the at least one generator, a communication connection for example with a control unit, a user interface unit, or a data storage. The applicator may further be coupled with a cooling unit, for example via a fluid connection, and/or may be in fluid coupling with a fluid delivery unit. A distal part (204) of the applicator may include a portion suitable for engagement with at least part of a human body for example with a skin and may include at least one energy delivery element (203).

The applicator as a whole, or at least part of the applicator, may be replaceable or disposable. For example, the distal part (204) of the applicator or at least part of it may be replaceable or disposable, for example the part suitable for engagement with at least part of a human body including the energy delivery elements. The replaceable part is any portion of the distal part which is detachable from the applicator and may be replaced. The replaceable part may be for example suitable for sterilization and reuse or the part may be disposable which means for example it may be adapted for a single use only. The replaceable part may be coupled to the rest of the applicator, wherein the coupling may include a mechanical means, for example a locking mechanism, snapping mechanism (e.g. pin and hole, snap fit, hook and hole), screwing mechanism (for example thread or screws), bayonet and so on. The coupling may include magnets as well. The electrical lead between the replaceable part (and thus the at least one energy delivery element) and the rest of the applicator should be adapted for the replacement of the replaceable part. The electrical lead may include for example at least one connector adapted for a connection and a disconnection during a coupling and a decoupling of the replaceable part. The connector may be for example a kind of deflectable connector, for example a spring loaded connector, or another connector allowing secure and easy disconnection and connection during a coupling and a decoupling of the replaceable part, for example a magnetic connector. There may be one common connector, or there may be plurality of connectors between the replaceable part and the rest of the applicator. For example there may be one common connector adapted for a connection of RF signal lead and PEF signal lead, or one or more (e.g. 1-50 or 2-40 or 3-20 or 4-15) of the RF signal leads and one or more (e.g. 1-20 or 2-16 or 3-14 or 4-12) of the PEF signal leads may have different connectors.

In some instances, the applicator may include some of the other parts of the system, for example it may include at least one generator, at least one control unit, at least one user interface unit, a memory, at least one cooling unit and/or at least one fluid delivery unit. It may further include for example a mechanism for an extension/retraction of the at least one energy delivery element, for example a mechanism for a manual extension/retraction or for a motorized extension/retraction, for example a motor. The applicator may further include at least one sensor, for example a temperature sensor, position sensor or impedance sensor.

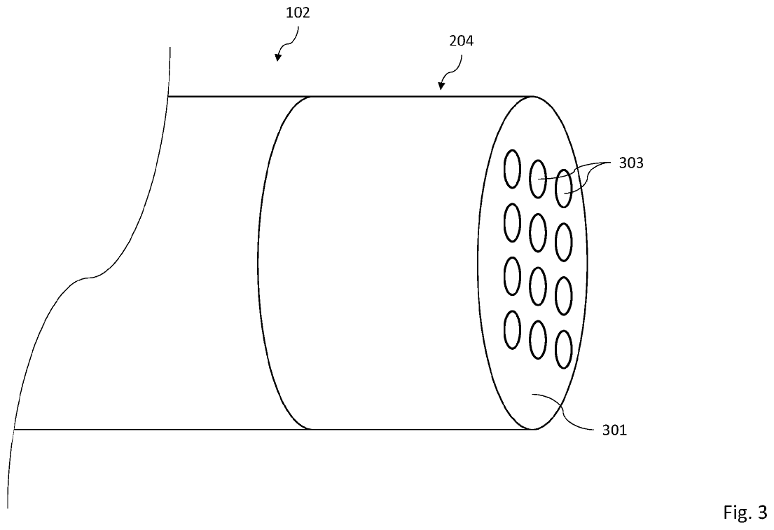

In FIG. 3 is shown an example of a distal part (204) of the applicator (102) suitable for engagement with at least part of a human body for example with a skin. The distal part includes a number of energy delivery elements, in this particular example superficial energy delivery elements (303) for example in the form of flat energy delivery elements or superficial pins. The energy delivery elements may be all of one type, for example configured to deliver RF field and PEF, or there may be multiple types of energy delivery elements, some adapted for delivery of RF field and others adapted for delivery of PEF. In another aspect some of the energy delivery elements may be suitable for delivery of both RF field and PEF and some others may be suitable for delivery of only one of RF field or PEF. The number of energy delivery elements may be from 4 to 200, or from 6 to 120, or from 10 to 80. The maximal cross-section area of the energy delivery element may be for example from 0.05 mm² to 5 mm². The surface of the energy delivery element may be for example from 0.05 mm² to 10 mm² . In examples where the energy delivery elements are superficial pins, the maximal diameter of the pins may be for example from 0.05 mm to 3 mm. The distance between particular energy delivery elements may be from 0.5 mm to 10 mm. All of the above stated dimensions may be uniform or may differ within one applicator. The surface (301) of the distal part of the applicator comprising the energy delivery elements may be flat or may be shaped (e.g. curved) for example to conform to a specific part of a human body anatomy.

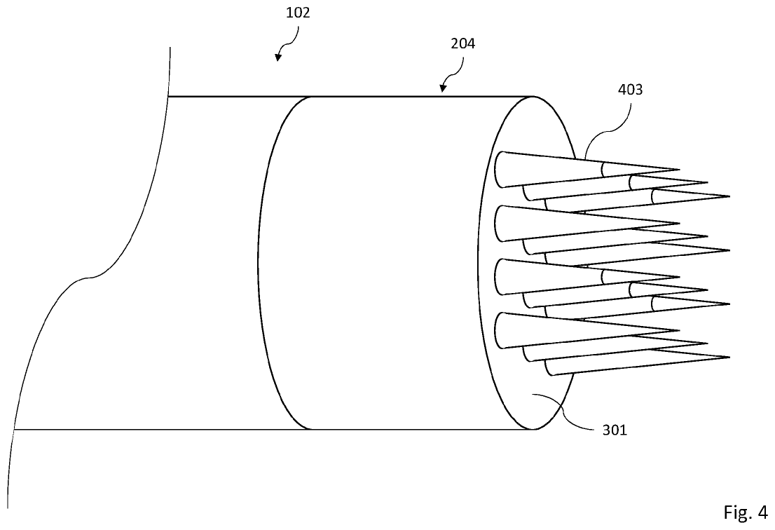

In FIG. 4, another example is shown of a distal part (204) of the applicator (102) suitable for engagement with at least a part of a human body for example a skin. The depicted example includes a number of energy delivery elements, in this particular example invasive energy delivery elements (403) for example in the form of needles. The energy delivery elements may be of one type, for example suitable for delivery of RF field and PEF, or there may be more types of energy delivery elements, some adapted for delivery of RF field and others adapted for delivery of PEF. In another aspect, some of the energy delivery elements are suitable for delivery of both RF field and PEF and some others may be suitable for delivery of either RF field or PEF only. The number of energy delivery elements may be from 4 to 200, or from 6 to 120, or from 10 to 80.

The length of the at least one energy delivery element may be fixed or may be adjustable (the energy delivery element may be for example extendable from at least one surface of the applicator), hence a depth of penetration of the energy delivery element into a tissue may be fixed or may be adjustable. In aspects with plurality of energy delivery elements where the length of the energy delivery elements is fixed, all of the energy delivery elements may have the same length or the energy delivery elements may vary in length. The length may vary for example from 0.1 mm to 20 mm, or from 0.2 mm to 15 mm or from 0.3 mm to 10 mm. In examples where the length of the at least one energy delivery element is adjustable, the range of possible lengths of the extended energy delivery element (extension length measured from the tip of the energy delivery element to a surface of the applicator from which is the energy delivery element extended) may be for example adjusted from 0.1 mm to 20 mm, or from 0.2 mm to 15 mm or from 0.3 mm to 10 mm. The length or the extension of the energy delivery element may be chosen according to intended use of the device and/or intended area of the body where the device should be used, for instance by way of following non-limiting examples. For example energy delivery elements extended to a length or having a length from 0.1 mm to 0.8 mm may be used for skin rejuvenation, apoptosis of senescent cells, melasma treatment, hyperpigmentation treatment, PIH treatment and/or rosacea treatment in an area of face of a patient. Extension or a length of the energy delivery element from 0.4 mm to 3 mm may be used for skin rejuvenation, apoptosis of senescent cells, wrinkles reduction, skin tightening, stretch marks reduction, melasma treatment, rosacea treatment and/or hyperhidrosis treatment in an area of face and/or body of a patient. Energy delivery elements retracted or with length from 3 mm to 10 mm may be for example used in a body of a patient for wrinkles reduction, senescent cells apoptosis, skin tightening, stretch marks reduction, scars treatment, fat reduction and/or cellulite reduction.

In examples with extendable energy delivery elements it may be desirable to be able to limit the maximum possible extension length of the energy delivery elements. It may be desirable for example for safety reasons. An example of such a situation may be usage of the device on a specific part of a human body where a needle extension beyond specific distance could be unpleasant for a patient or could possibly cause harm or unwanted side effects. For example an operator may want to limit maximal length of the extended energy delivery elements while treating patient in a face area. The length (extension) and/or a maximum length (extension) may be for example set by operator via a user interface unit. This setting may be executed and controlled for example by a control unit. However, for a further safety the device may for example include adjustable mechanical barrier, for example in the applicator, preventing a extension of the energy delivery elements beyond certain, predefined maximal extension length. For instance, for a face treatment the barrier may be set to prevent the extension of the energy delivery elements beyond length of 1 mm-to 5 mm (for example to 2 mm). For treatment of the body the barrier may be set to prevent the energy delivery extension beyond a length of 2 mm to 8 mm (for example to 4 mm). The barrier may be set for example by setting device for example knob, dial, lever or similar device, for example placed on the applicator. The setting device may be continuously adjustable or may be adjustable in steps and/or may include several predefined positions and markings identifying at least one predetermined maximal extension length. The barrier may include for example a mechanical stopper.

In examples with plurality of energy delivery elements the distance between particular energy delivery elements may be from 0.5 mm to 10 mm, and it may be uniform or it may differ within an applicator. The invasive energy delivery elements in the form of needles may have a diameter for example from 0.05 mm to 2 mm or from 0.1 mm to 1 mm or from 0.15 mm to 0.5 mm and again may be uniform or may differ within an applicator. The surface (301) of the distal part of the applicator including the energy delivery elements may be flat or may be curved for example to conform to a specific part of a human body anatomy.

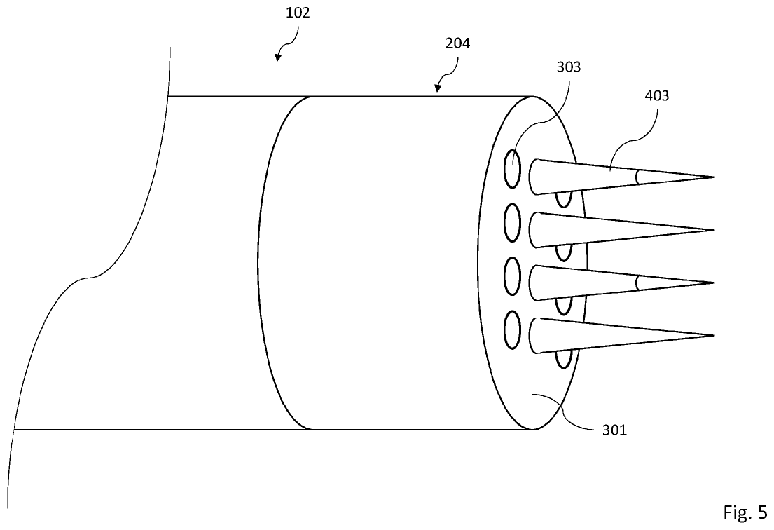

As stated above, in some examples the at least one energy delivery element may be adapted either for delivery of both RF field and PEF, or they may be particularly adapted for delivery of one of RF field or PEF. In various examples the plurality of energy delivery elements may be physically the same (may have the same shape, constitution, size, chemical composition), or the physical form of the energy delivery elements may differ (at least one of a shape, constitution, size or chemical composition may differ). In aspects with different energy delivery elements they may be included in one applicator and/or in different applicators, for example FIG. 5 shows an applicator (102) with a distal part (204) of the applicator including both invasive energy delivery elements (403) and superficial energy delivery elements (303).

All of the energy delivery elements may have the same electrical coupling to the at least one generator and for example at least one control unit may ensure the right signal is distributed to the correct energy delivery element at the right time. Different energy delivery elements may however have separate, different couplings to the at least one generator. For example the at least one energy delivery element suitable for a delivery of RF field may have a separate coupling to at least one generator suitable for generating the RF signal and/or an energy delivery element suitable for delivery of PEF may have a separate coupling to the at least one generator suitable for generating of PEF signal. In some aspects at least one energy delivery element suitable for delivery of PEF may have different physical form than the at least one energy delivery element suitable for delivery of RF field.

In aspects where at least one energy delivery element is invasive and extendable and/or has an adjustable length, the extension and/or the retraction of the energy delivery element may be driven manually or may be motorized. The mechanism enabling the extension and/or the retraction may be included in the applicator, for example in the proximal part of the applicator. The extension and/or the retraction may be controlled by a control unit and may be set for example via a user interface unit. One applicator may comprise at least one fixed energy delivery element and at least one of extendable and/or adjustable energy delivery element, or it may include only at least one fixed or only at least one extendable and/or adjustable energy delivery element. In aspects where the applicator includes more than one extendable and/or adjustable energy delivery element, the control of the extension and/or the retraction may be common for all of them or they may be controlled (extended and/or retracted) individually, or for example the energy delivery elements may be organized in groups and the extension and/or retraction may be controlled by the groups of energy delivery elements. For example at least one energy delivery element adapted for delivery of RF field may be controlled independently on the at least one energy delivery element adapted for delivery of the PEF.

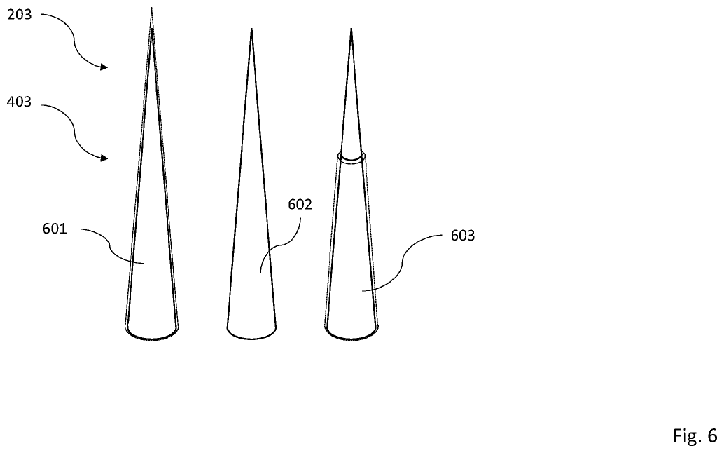

The at least one energy delivery element (203) may be manufactured from different materials suitable for delivery of RF field and/or PEF to, or adjacent to, at least part of a human body. An example of such a material may be copper, gold, steel, titanium, platinum, platinum-iridium, silver and so on. The at least one energy delivery element (203) may be insulated (601), uninsulated (602) or partially insulated (603) as shown in FIG. 6. In examples where there is more than one energy delivery element included on the applicator, any combination of insulated, uninsulated or partially insulated energy delivery elements may be possible. For example at least one energy delivery element suitable for delivery of RF energy may be uninsulated or partially insulated and at least one energy delivery element suitable for delivery of PEF may be insulated, partially insulated or uninsulated. In aspects where at least one energy delivery element (203) is invasive energy delivery element (403) and has a form of a needle, partial insulation may mean the tip of the needle is without insulation while the rest of the needle is insulated. The uninsulated portion of the tip may take up several percent of the needle's overall length, for example from 0.1% to 20% or from 0.5% to 15% or from 1% to 10%. However partial insulation may also mean that other parts of the needle than the tip may be uninsulated. For example the needle may have several portions with insulation and several portions without insulation for example along the length of the needle. The insulation of the energy delivery elements may be achieved by any suitable material, for example by polyimide, polyurethane, polyester, polyvinylchloride (PVC), rubber, rubber-like polymers, nylon, polyethylene, polypropylene, silicone, fiberglass, ceramic, ethylene propylene diene monomer (EPDM), different fluoropolymers like polytetrafluoroethylene (PTFE) and so on. In some examples, a combination of different insulating materials may be possible. For example in examples having an invasive energy delivery element, a tip portion may be coated with a hard material, like ceramic which can withstand damage due to wear and tear, while the rest of the energy delivery element may be coated with a different material, for example polyimide, polyurethane, polyester, polyvinylchloride (PVC), rubber, rubber-like polymers, nylon, polyethylene, polypropylene, silicone, fiberglass, ethylene propylene diene monomer (EPDM), or different fluoropolymers like polytetrafluoroethylene (PTFE).

In examples with a plurality of energy delivery elements included in the applicator they may be arranged for example in rows or in circles, or they may be seemingly disorganized. Together they may form an array. An exemplary array of energy delivery elements may include from 2 rows to 100 rows, with from 2 energy delivery elements to 100 energy delivery elements in a row. One example of the array of delivery elements may include for example two rows of the energy delivery elements, where each of the row includes 2-4 energy delivery elements. Distance between the rows may be from 2 mm to 8 mm and distances between the energy delivery elements in one row may be from 1.5 mm to 7 mm. In another example an array or energy delivery elements may include 3-8 rows of 3-8 energy delivery elements. The distances between rows and energy delivery elements in the rows may be for example from 1 mm to 4 mm.

An example of a layout with energy delivery elements organized in circles may include from one circle to 20 circles of the energy delivery elements. Each circle may for example include from 3 to 100 energy delivery elements. There may be for example one or more energy delivery elements placed in a center of the at least one circle. The at least one energy delivery element placed in the center may have for example a different polarity than the energy delivery elements placed in the at least one circle.

One method of treatment of a human body according to this disclosure includes a delivery (application) of RF energy and PEF into at least part of a human body for example by a system described above. The part of a human body may be for example a skin, particularly for example a skin tissue, for example, at least one of a hypodermis, a dermis, an adipose tissue and a mucosal tissue. The method may be used for example for treatment of fine lines and wrinkles, acne and acne scars, scars, stretch marks, fat reduction, hyperhidrosis, age spots/hyperpigmentation, post-inflammatory hyperpigmentation (PIH), melasma, rosacea, warts, skin tags or tumors.

The RF energy may be delivered in a monopolar, a bipolar and/or a multipolar mode to create thermal damage in the treated tissue. Frequencies of the RF field delivered to the tissue may be in the range of 80 kHz to 5 GHz, or in the range of 0.1 MHz to 500 MHz or in the range of 0.2 MHz to 50 MHz, or in the range of 0.3 MHz to 30 MHz. The delivery of the RF field may be provided by at least one RF pulse (701) having a specified time (t1), referred as a RF pulse duration, which may be in the range of 0.1 ms to 2500 ms, or in the range of 0.5 ms to 2000 ms, or in the range of 1 ms to 1500 ms. The current density of the radiofrequency field delivered by one energy delivery element may be in the range of 1 A/cm² to 300 A/cm², or in the range of 5 A/cm² to 200 A/cm², or in the range of 10 A/cm² to 150 A/cm². An RF pulse voltage (amplitude) may be from 1V to 1000V. A maximum energy may be dependent on the type of the energy delivery element and may for example be between 10 mJ and 500 mJ per needle-type energy delivery element or pin-type energy delivery element.

In a monopolar mode, the RF energy may be delivered among one or more energy delivery elements placed on the applicator and one or more indifferent electrodes placed in the distance, for example on the skin of the patient and/or for example on the applicator. The one or more indifferent electrodes may have in some aspects a significantly larger surface area than the sum of the surface areas of the active energy delivery elements. In a bipolar mode the RF energy is applied between two or more, for example adjacent energy delivery elements with different polarities. In this example the sum of the surfaces of the active energy delivery elements with the first polarity is similar to the sum of the surfaces of the active electrodes with the second polarity. In a multipolar mode there is a first at least one energy delivery element operating in a mode with a first polarity and a second at least one energy delivery element operating in a mode with a different polarity (which may be an opposite polarity) than the operating mode of the first at least one energy delivery element. A surface or a sum of the surfaces of the first at least one energy delivery element is significantly smaller than a surface or a sum of the surfaces of the second at least one energy delivery element.

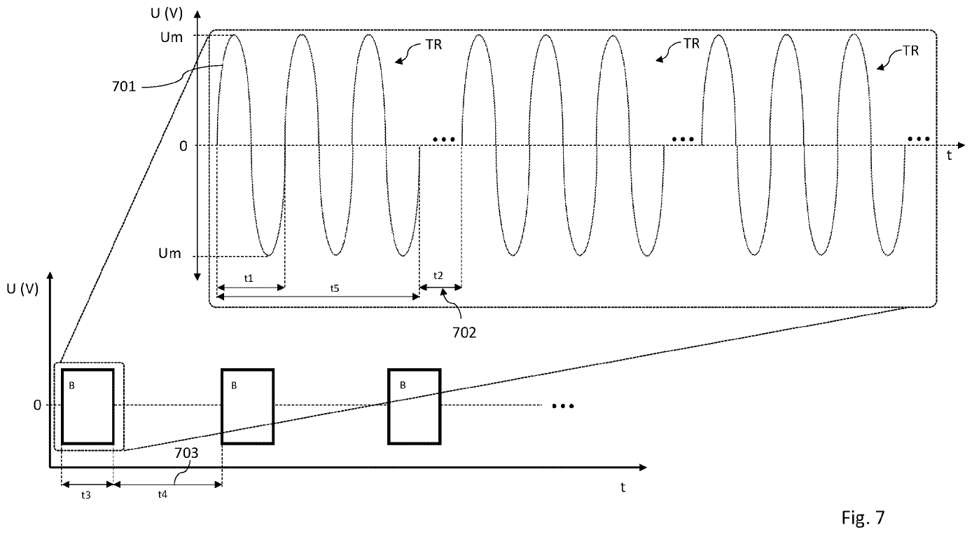

FIG. 7 shows an example of an RF treatment protocol. The signals for the RF pulses may be generated in the form of RF signals with an RF generator (a generator suitable for generating RF signals) and may be delivered to the target tissue in the form of an RF field by the at least one energy delivery element which may be included on the applicator and which may be electrically coupled to the RF generator. The RF pulses (701) may be delivered as single RF pulses (701), with an amplitude Um and a pulse width with a time duration t1, or they may be repeated in at least one train (TR) with a time duration t5, where parameters of the pulses may vary or may remain constant. The trains of pulses may be repeated as well and may create a burst (B). Particular trains (TR) may be separated by an inter-train pause (702) with time duration t2. The burst (B) may have a time duration t3 and in examples with a plurality of bursts (B) they may be separated by an inter-burst pause (703), defined by time period t4. The RF pulses may be delivered continuously, for example in one train during one applicator application without pauses between particular pulses, or they may be delivered in sequential manner. In the sequential delivery single RF pulses (701) may be separated from each other by pauses of non-delivery with a certain time duration. Another option of sequential delivery may include a delivery of several trains (TR) of pulses separated by an inter-train pause (702).

The PEF may be delivered (applied) in a monopolar, a bipolar or a multipolar mode. It may be delivered in the form of electric pulses including monophasic (single polarity) pulses, symmetrical and/or asymmetrical biphasic pulses. The pulses may repeat from 1× to 100000× during a treatment or from 1× to 1000× or from 1× to 100× or from 1× to 50× during one applicator application. The frequency of the high frequency pulses may vary from 1 Hz to 500 Hz or from 5 Hz to 250 Hz or from 10 Hz to 100 Hz. An amplitude (Um) of the monophasic pulses may vary from 10 V up to 15 kV, and the peak to peak amplitude of biphasic pulses may vary from 20 V to 30 kV.

In a monopolar mode, an electric field for example PEF may be created between one or more energy delivery elements included on the applicator and one or more indifferent electrodes placed elsewhere, for example on the skin of the patient and/or for example on the applicator. The one or more indifferent electrodes may have in some aspects a significantly larger surface area than the sum of the surface areas of the active energy delivery elements.

In bipolar mode the electric field is created between two or more, for example adjacent energy delivery elements with different polarities. In this example, the surface area or the sum of the surface areas of the one or more energy delivery elements with the first polarity is similar to a surface area or a sum of the surface areas of the one or more energy delivery elements with the second polarity. The energy delivery elements with different polarities in the bipolar mode may be all surface energy delivery elements, all invasive energy delivery elements, or in some examples at least one energy delivery element having a first polarity may be a surface energy delivery element and at least one other energy delivery element having a different polarity than the first polarity may be an invasive energy delivery element.

In a multipolar mode there is at least one first energy delivery element operating in a mode with first polarity and at least one second energy delivery element operating in a mode with a different polarity (which may be an opposite polarity) than the operating mode of the at least one first energy delivery element. A surface area or a sum of the surface areas of the at least one first energy delivery element may be significantly smaller than a surface area or a sum of the surface areas of the at least one second energy delivery element. The ratio between the surfaces may be from 2:3 to 1:100, or 3:5 to 1:70, or 1:2 to 1:40. The energy delivery elements with different polarities in the multipolar mode may be all surface energy delivery elements, all invasive energy delivery elements, or at least one energy delivery element having a first polarity may be a surface energy delivery element and at least one other energy delivery element having a different polarity than the first polarity may be an invasive energy delivery element.

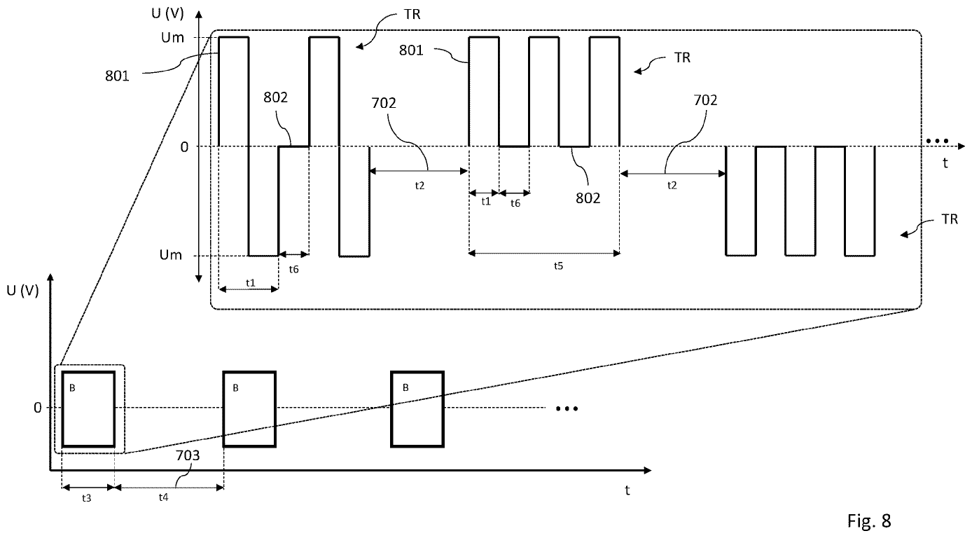

FIG. 8 is an example of a PEF treatment protocol. The signals for the PEF pulses may be generated in the form of PEF signals by an PEF generator (a generator suitable for generating PEF signals) and may be delivered to the target tissue in the form of an PEF by the at least one energy delivery element which may be included on the applicator and which may be electrically coupled to the PEF generator. The PEF treatment protocol may include at least one or a series of PEF pulses (801) and pauses (702, 703, 802). The PEF pulses (801) may be further organized in units with a certain hierarchy like trains (TR) and bursts (B). The PEF pulse (801) may be defined for example by a shape, an amplitude (Um) with a certain voltage and a pulse width with a time duration t1. The pulse amplitude (Um) may be either negative or positive (the pulse may have a negative voltage or a positive voltage) in the case of monophasic pulses. The PEF pulses may be separated from each other by an inter-pulse pause (802), which may be defined by a time duration (t6) and a voltage. The voltage during the inter-pulse pause may drop to OV or it may have a positive or negative voltage value. The absolute voltage value of the inter-pulse pause is smaller than an absolute voltage (amplitude (Um)) of the adjacent electrical pulse, particularly up to 50% of the amplitude (Um) of the adjacent electrical pulse. In situations where the electrical pulse has a positive amplitude (Um), the voltage value of the inter-pulse pause may stay positive between OV and the electrical pulse amplitude (Um), and in situations where the electrical pulse has a negative amplitude (Um), the voltage value of the inter-pulse pause may stay negative, between OV and the electrical pulse amplitude (Um). Biphasic pulses may be symmetrical or asymmetrical in at least one of time, amplitude or energy.

The train (TR) of PEF pulses (801) may be characterized by a time duration t5. Parameters of the pulses included in the train may vary or may remain constant. The train (TR) may include monophasic pulses, biphasic pulses or both. The trains of pulses (TR) may be repeated and may create a burst (B). Particular trains (TR) may be separated by an inter-train pause (702) with time duration t2. The burst (B) may have a time duration t3 and in examples with a plurality of bursts (B) they may be separated by an inter-burst pause (703), defined by time period t4.

The PEF pulses may be combined with extra pre-pulses for example for tissue conditioning. Pulses may be delivered as single pulses or they may be repeated in at least one train, where parameters of the pulses may vary or may remain constant. Trains of pulses may be repeated as well and may create one or more bursts. A maximal amplitude of the pulses may depend on the target tissue, electrode size and/or electrode distance in order to create an electric field with a maximum electric field magnitude for example between 0.1 kV to 30 kV or between 0.2 kV to 5 kV or between 0.3 kV to 2 kV per cm in a target tissue. A duration of the pulse may vary from a nanosecond range to milliseconds range, for example from 2 ns to 10 ms, or from 10 ns to 1 ms or from 100 ns to 100 μs. The shape of the pulse may be for example a square, a curve similar to exponential discharge, a rectangle, a saw, a triangle, trapezoidal or a sinusoidal. Maximal power output may be 0.01 W to 500 W. The energy of the pulse may be from 0.01 J to 1 J or from 0.05 J to 0.7 J or from 0.1 J to 0.45 J. Energy delivered per area may vary from 0.01 J/mm² to 1 J/mm² or from 0.05 J/mm² to 0.7 J/mm² or from 0.1 J/mm² to 0.45 J/mm² . Energy delivered per volume may be from 0.01 J/mm³ to 1 J/mm³, or from 0.05 J/mm³ to 0.6 J/mm³, or from 0.1 J/mm³ to 0.3 J/mm³.

The parameters of the PEF pulses may not be fixed (fixedly set) to one value, but may be changed according to a treatment, for example before a start of a treatment or during a treatment. In some examples the parameters of the PEF pulses need to be changed, or set for example in case the device is adapted to use multiple applicators and/or multiple distal parts of the applicator, that can be exchanged (replaced) before, during or after the treatment. The replaceable applicators or distal parts of applicator may be of several types, including different energy delivery elements, for example some of the applicators or distal parts may include only superficial energy delivery elements, some of them may include superficial and invasive energy delivery elements and some of them may include invasive energy delivery elements only. Other kinds of the applicators or distal parts of the applicator may include insulated energy delivery elements, some of them may include uninsulated energy delivery elements or partially insulated energy delivery elements and some of the applicators or distal parts of the applicator may use more than one type of insulated/partially insulated / uninsulated energy delivery elements. In another example the applicator or distal part of the applicator may include different number of energy delivery elements, or energy delivery elements with different dimensions, or energy delivery elements with different distances between them, or combination of the different number of elements with different dimensions or with different distances between them.

In examples when the device includes plurality of the exchangeable applicators or distal parts of the applicator with differences stated above, it may be necessary to adapt the PEF pulse parameters and/or a pulse protocol to each applicator or distal part of the applicator, for example to prevent possibility of creation of sparks during application of the PEF pulses. The PEF pulse parameters and/or a pulse protocol parameters may need to be set manually by operator, for example via user interface unit, or they may be set automatically, for example after coupling of the particular applicator or applicator distal tip to the device. In examples where PEF pulse parameters are set automatically, particular applicators or applicator distal tips may include information about its configuration (kind of applicator) and the device may be adapted to receive the information and to set the parameters accordingly. The information may be for example stored in a memory included in the applicator or distal tip of the applicator and communicated for example to the control unit via electrical coupling.

The adjustable parameters of the PEF pulse and/or the pulse protocol may include for example amplitude, pulse width, number of pulses, length of inter pulse pauses, length of inter train pauses and so on. The parameters may be set for example based on a distance between particular energy delivery elements, for example energy delivery elements creating a bipolar pair. In particular examples of applicator or distal tip of the applicator including invasive energy delivery elements, for example in form of needles, the maximal amplitude may be need to be restricted. For example maximal absolute maximal amplitude of the PEF pulse according to distance between needles in bi-polar pair may be restricted in following way-for needle distance of 3 mm to 6 mm, maximal amplitude may be set to 3000 V to 6000 V, for distance of 2 mm to 3 mm, maximal amplitude may be set to 2000 V to 3000 V, for distance of 1.5 mm to 2 mm, maximal amplitude may be set to 1300 V to 2200 V, for distance for 1 mm to 1.5 mm, maximal amplitude may be set to 1000 V to 1700 V.

Although the terms “electric fields” or “pulsed electric fields” are mentioned in the description, electric fields as contemplated herein may further comprise a magnetic component.

A method of treatment of at least part of a human body according to this disclosure includes an application of the RF field and the PEF during one treatment. The RF field and the PEF may be applied to the tissue of at least part of a human body by a system or a device described above. The RF field and the PEF may be applied during one or more applications of the at least one applicator or during a treatment via at least one energy delivery element, which may be included on the at least one applicator.

A treatment protocol of delivery of the RF field and PEF may include at least one RF pulse and at least one PEF pulse. In the case of a plurality of pulses, they may be applied in trains and bursts as well. The pulses, trains and bursts may be combined. The RF pulses may be for example delivered with the PEF pulses in an alternating manner. The same can apply to the trains and/or bursts. Trains and/or bursts with the RF pulses may alter with trains and/or bursts with the PEF pulses. In other aspects the pulses, trains of pulses or bursts of one energy (either RF or PEF) may be delivered in a first time period and the pulses, trains or bursts of the second energy may be delivered in a second time period during the applicator application or during the treatment. The time periods may be separated, may build on each other or they may overlap. Single pulses of one energy (either RF or PEF) may not only be combined with single pulses of the other energy, but also with trains or bursts of pulses of the other energy. For example a single RF pulse may be combined with a train of PEF pulses, or a single PEF pulse may be combined with a train of RF pulses.

The treatment protocol may include extra pre-pulses applied before application of the treatment RF pulses or PEF pulses. The pre-pulses may serve for example for tissue conditioning or for measurement purposes. Conditioning pre-pulses may for example cause pre-heating of the target tissue to non-coagulative temperatures. Measurement pre-pulses may for example serve for current measurement or for measurement of impedance of the target tissue. The pre-pulses may include RF pulses and/or PEF pulses.

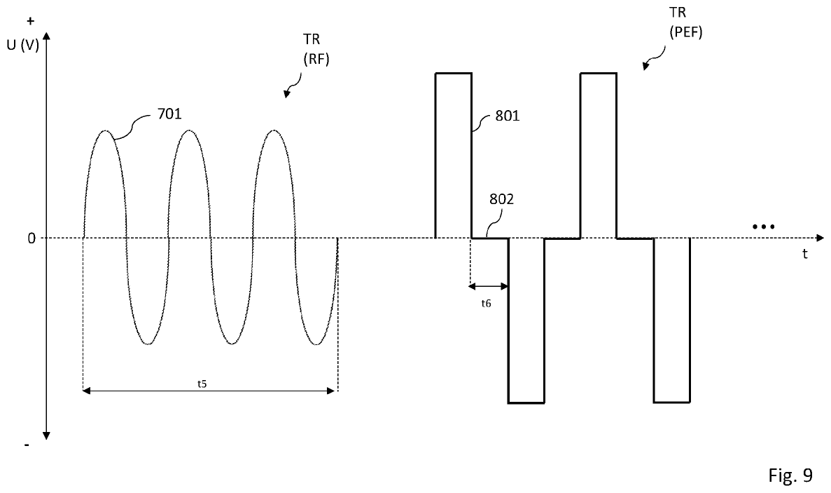

An example of a combination of a train (TR(RF)) of the RF pulses (701) with a train (TR (PEF)) of PEF pulses (801) in one treatment protocol may be seen in FIG. 9. Such an exemplary treatment protocol may start with a delivery of the RF pulses (701), for example at least one train (TR(RF)) of RF pulses for a first time period (15), which may last for example from 0.01 s to 10 s or from 0.05 s to 5 s or from 0.1 s to 1 s. The RF pulses (701) within the train (TR(RF)) may be delivered in a continuous manner. After the delivery of the RF pulses (701) follow a delivery of the PEF pulses (801), for example a specific number (for example 1-100) of the PEF pulses (801). Particular PEF pulses (801) may be separated from each other by an inter-pulse pause (802), which may be defined by a time duration (t6). The described delivery of the RF pulses (701) followed by the PEF pulses (801) may be delivered during one applicator application. The treatment protocol delivered during the one applicator application may further include delivery of pre-pulses before the application of the treatment RF pulses (701) and/or the PEF pulses.

In another example of a combination of a train (TR(RF)) of the RF pulses (701) with a train (TR (PEF)) of PEF pulses (801) in one treatment protocol, the RF pulses may be delivered in more than one train (TR(RF)) of the RF pulses. In this example each train may include at least one RF pulse (701) and the trains (TR(RF)) of the RF pulses may be separated by an inter-train pause (702).

The “application of the at least one applicator” or the “applicator application” should be understood as a specific term in comparison with the general term “application”. It means specifically an act of a delivery of the applicator adjacent to the at least part of a human body and executing at least one energy delivery adjacent to a tissue of the at least part of a human body before removing the at least one applicator or before relocating it into a different location. The treatment means one or more applications of the at least one applicator either at the spatially same or different locations of at least a part of a human body. The treatment may be started for example by turning the device on and may include at least one application of the at least one applicator and may be finished for example by turning the device off.

Both energies may be applied invasively or non-invasively, for example superficially (for example by superficial energy delivery elements), or one of the energies may be applied non-invasively, for example superficially (for example the PEF) and the other one may be applied invasively (for example the RF field) during a treatment or during one application of the at least one applicator. The invasive application may be carried out by invasive energy delivery elements. Non-invasive, for example superficial application of the RF field and invasive application of the PEF during one application of the at least one applicator or one treatment is also possible. An option that at least one of the energies is applied both non-invasively and invasively during one application of the at least one applicator or during one treatment is also contemplated.

The application of the at least one applicator or a treatment may include a non-invasive application of the RF field and/or the PEF. The RF field and/or the PEF may be in this example delivered via at least one superficial energy delivery element placed adjacent to the tissue. The energy delivery element in this example does not penetrate the tissue before, during or after the application of the at least one applicator or a treatment.

In another example the RF field may be applied during an application of the at least one applicator or a treatment invasively. In one aspect, one or more RF pulses may be applied for example via invasive energy delivery elements. The energy delivery elements may be included on the applicator and may have one or more fixed lengths. In this example the applicator may be brought adjacent to a tissue, and a pressure sufficient for the at least one energy delivery element included on the applicator to at least partially penetrate into the tissue may be applied to, or against the applicator. The one or more RF pulses may be delivered into the tissue during or after the at least partial insertion of the at least one energy delivery element into the tissue. In one example the one or more RF pulses may be delivered after an insertion of the at least one energy delivery element into a specified depth. The one or more RF pulses may be followed by application of one more other RF pulses. The first one or more RF pulses may have a different RF pulse duration and/or amplitude than the other one or more RF pulses. In another example the applicator may be brought adjacent to a tissue, the one or more RF pulses may be delivered into the tissue and may be followed by an application of one or more other RF pulses. During or after the delivery of any of the RF pulses the at least one energy delivery element may be at least partially inserted into the tissue. The first one or more RF pulses may have different RF pulse duration and/or amplitude than the other one or more RF pulses.

The energy delivery elements may also in some examples be extendable from at least one surface of the applicator. In this example of an application, the applicator may be applied adjacent to a tissue and the energy delivery elements may be extended a predefined length from the at least one surface of the applicator such that at least a portion of one or more of the energy delivery elements at least partially penetrate the tissue or are inserted into the tissue.

After the at least partial insertion of the at least one energy delivery element into a specified depth, one or more RF pulses may be delivered into the tissue. The one or more RF pulses may be followed by application of one more other RF pulses. The first one or more RF pulses may have a different RF pulse duration and/or amplitude than the other one or more RF pulses.

The application of the at least one applicator or a treatment may include a sequential application of RF field. In one application of the at least one applicator, more than one RF pulse may be applied to the tissue. In one example of an invasive treatment, one or more RF pulses may be applied in first penetration depth of the one or more invasive energy delivery elements, then the one or more energy delivery elements may be repositioned deeper and/or shallower within a tissue (for example the length of the energy delivery elements may be reconfigured) and another one or more RF pulses may be applied by the one or more repositioned invasive energy delivery elements into the second penetration depth.

In another example the PEF may be applied during an application of the at least one applicator or a treatment invasively. In one aspect, one or more PEF pulses may be applied for example via invasive energy delivery elements. The energy delivery elements may be included on the applicator and may have one or more fixed lengths. In this example the applicator may be brought adjacent to a tissue, and a pressure sufficient for the at least one energy delivery element included on the applicator to at least partially penetrate into the tissue may be applied to, or against the applicator. The one or more PEF pulses may be delivered into the tissue during or after the at least partial insertion of the at least one energy delivery element into the tissue. In one example the one or more PEF pulses may be delivered after an insertion of the at least one energy delivery element to a specified depth. The one or more PEF pulses may be followed by application of one more other PEF pulses. The first one or more PEF pulses may have a different PEF pulse duration and/or amplitude than the other one or more PEF pulses. In another example the applicator may be brought adjacent to a tissue, the one or more PEF pulses may be delivered into the tissue and may be followed by an application of one more other PEF pulses. During or after the delivery of any of the PEF pulses the at least one energy delivery element may be at least partially inserted into the tissue. The first one or more PEF pulses may have a different PEF pulse duration and/or amplitude than the other one or more PEF pulses.

The energy delivery elements may also in some examples be extendable from at least one surface of the applicator. In this example of an application the applicator may be applied adjacent to a tissue, the energy delivery elements may be extended a predefined length from the at least one surface of the applicator such that at least a portion of one or more of the energy delivery elements at least partially penetrate or are inserted into the tissue. After the at least partial insertion of the at least one energy delivery element into a specified depth, one or more PEF pulses may be delivered into the tissue. The one or more PEF pulses may be followed by application of one more other PEF pulses. The first one or more PEF pulses may have a different PEF pulse duration and/or amplitude than the other one or more PEF pulses.

The application of the at least one applicator or a treatment may include a sequential application of PEF. In one application of the at least one applicator, more than one PEF pulse may be applied to the tissue. In one example of an invasive treatment, one or more PEF pulses may be applied in first penetration depth of the one or more invasive energy delivery elements, then the one or more energy delivery elements may be repositioned deeper and/or shallower within a tissue (for example the length of the energy delivery elements may be reconfigured) and another one or more PEF pulses may be applied by the one or more repositioned invasive energy delivery elements in the second penetration depth.

Advantageously both the RF field and the PEF may be applied into the tissue of at least part of a human body during at least one application of the at least one applicator or during at least one treatment. RF field and PEF may both be applied simultaneously or partially simultaneously or in different time periods during at least one application of the at least one applicator or during at least one treatment.

In one example the RF field may be for example applied during one application of the at least one applicator and the PEF may be applied during another application of the at least one applicator, while both of the applications are applied in different time periods, which may be time-separated.

In examples where PEF and RF field are applied by different applicators, the delivery of the PEF may happen in a first application of the at least one applicator and the delivery of the RF field may take place in a second application of the at least one applicator, wherein the first and the second applications may take place in the same time period, the time periods of the application may at least partially overlap each other, or they may be time-separated.

In another example the RF field may be for example applied during one time period and the PEF may be applied during another time period, while both time periods take place during one application of the at least one applicator. The time periods taking place during one application of the at least one applicator may be completely time-separated or may at least partially overlap each other.

In a further aspect, RF field and PEF may be applied in a sequential manner. The sequence of the delivery of the RF field and the PEF may happen during one application of the at least one applicator and/or during the treatment. The sequence of the delivery means the time period of the delivery of the RF field and/or the PEF may repeat more than once during the application of the at least one applicator or during the treatment. In this example the time periods of the delivery of the RF field and the PEF may be time-separated or may at least partially overlap each other.

RF field and PEF may be both applied in the same depth of the tissue, or they may be applied in different depths. The RF and the PEF may also be applied by energy delivery elements placed in the same depth of the tissue, for example they both may be applied superficially, non-invasively by superficial energy delivery elements or invasively by invasive energy delivery elements of the same length, or the RF field may be applied by at least one energy delivery element having a first length and the PEF may be applied by at least one energy delivery element having a second length, wherein the first length is different than the second length. The PEF and the RF field may be delivered into different tissue depths.

For example the PEF may be applied by at least one superficial energy delivery element or an invasive energy delivery element into a first issue depth, while the RF field may be applied by at least one superficial or invasive energy delivery element into a second depth, wherein the first depth and the second depth may be different.

In one example RF field may be delivered by at least one invasive energy delivery element and PEF may be delivered by at least one different invasive energy delivery element, while at least one of the invasive energy delivery elements may be extendable. The RF field and the PEF may be applied by at least one energy delivery element that is at least partially extended to a delivery length (a length in which an energy is delivered by the energy delivery element) or by at least one fixed energy delivery element (implicitly in delivery length). The RF or the PEF may be applied (simultaneously or in time-separated manner) while all of the energy delivery elements are at least partially extended in their delivery lengths.

In another aspect, RF field and PEF may be applied in a time-separated manner in different time periods. The first field (one of RF field and PEF) may be applied by at least one first extendable energy delivery element, the second field may be applied by at least one second extendable energy delivery element different than the first energy delivery element. The energy delivery element delivering a field (one of RF field and PEF) in a particular time period may be called an active energy delivery element, the energy delivery element not delivering a field (one of RF field and PEF)) in the particular time period may be called an inactive energy delivery element. In this aspect, the first energy is delivered when the at least one active energy delivery element, adapted for delivery of the first field (either the RF field or the PEF) is extended to its delivery length, while the at least one inactive energy delivery element adapted for a delivery of the second field (either RF field or PEF), different than the first field may be fully or at least partially extended to a different length than its delivery length. The second field (either RF field or PEF) is delivered in another time period, when the at least one energy delivery element adapted for a delivery of the second field may become an active energy delivery element and may be extended to its delivery length, and the at least one energy delivery element adapted for a delivery of the first field may be extended to a different length than its delivery length. In situations where more than one energy delivery element is adapted for delivery of either RF field or PEF, only a portion of the inactive energy delivery elements may be extended to a different length than its delivery length.

In a further aspect, the RF field and the PEF may be applied again in a time-separated manner in different time periods. The first field (one of the RF field and the PEF) may be applied by at least one first fixed or extendable energy delivery element, the second field (one of the RF field and the PEF) may be applied by at least one second fixed or extendable energy delivery element different than the first energy delivery element. In this aspect the first field (one of the RF field and the PEF) may be applied with all of the energy delivery elements being at their delivery lengths and the second field (different than the first field) may be applied while at least one active energy delivery element, adapted for a delivery of the second field being at its delivery length, and while at least one inactive energy delivery element is extended to a different length than its delivery length. In examples where more than one energy delivery element is adapted for delivery of either RF field or PEF, only a portion of the inactive energy delivery elements may be extended to a different length than their delivery length.

In another aspect during a time period of delivery of a field (one of the RF field and the PEF) while all of the energy delivery elements are at their delivery lengths, all of the energy delivery elements may be active and may deliver the particular field during the time period, or only a portion of the energy delivery elements may be active delivering the field, while another portion may be inactive.

An order of the time period of a field delivery with all of the energy delivery elements at their delivery lengths and the time period of an application of the field (one of the RF field and the PEF) while at least one active energy delivery element is at its delivery length, and while at least one inactive energy delivery element is extended to a different length than its delivery length, may vary. For instance, in some examples the first field with all of the energy delivery elements at their delivery lengths may be applied first, followed by at least one of the energy delivery elements at least partially extended and then application of the second energy may take place. In some examples, first field (one of the RF field and the PEF) may be applied while the at least one inactive energy delivery element is extended, then at least one energy delivery element may be extended to a delivery length and the second field may be applied, for example while all of the energy delivery elements are extended.

In another example RF field and PEF may be applied by the same energy delivery elements. In this example may be all of the energy delivery elements included on the applicator adapted for the delivery of the RF field and PEF. RF field and PEF may be applied for example both during one applicator application for example in separate times. In this particular example all of the energy delivery elements included on the applicator may apply RF field as well as the PEF during a treatment or during the applicator application. The RF field may be applied by all of the delivery elements simultaneously in one time period, the PEF may be applied by all of the delivery elements simultaneously in another time period. The time period of the RF field delivery may be separated from the time period of the PEF delivery, for example the RF field delivery time period may be followed by the PEF delivery time period. Both of the delivery time periods may take place within on applicator application.

1-20. (canceled)

21. A system for treating skin tissue comprising at least one of a hypodermis, dermis, or mucosal tissue, the system comprising:
one or more generators, the one or more generators configured to generate radio frequency (RF) signals and pulsed electric field (PEF) signals;
an applicator electrically coupled to the one or more generators, the applicator configured to deliver an RF field and a PEF to the skin tissue based on the RF signals and the PEF signals from the one or more generators, the applicator comprising a plurality of energy delivery elements configured to deliver the RF field and the PEF to the skin tissue; and
a control unit in communication with the one or more generators and/or the one or more applicators, the control unit configured to control the delivery of the RF field and the PEF to the skin tissue during a single application of the applicator;
wherein the RF field is configured to cause destruction of cells and non-cellular tissue contained in the skin tissue by heating; and
wherein the PEF is configured to cause electroporation of cell membranes contained in the skin tissue.

22. The system according to claim 21, wherein the RF field is configured to cause destruction of cells and non-cellular tissue in a first area of the skin tissue; and
wherein the PEF is configured to cause electroporation of cell membranes in a second area in the skin tissue.

23. The system according to claim 22, wherein the cells and the non-cellular tissue are destroyed by heating to a temperature of 43° C. to 120° C..

24. The system according to claim 22, wherein the second area intersects the first area.

25. The system according to claim 22, wherein the first area is in close proximity to at least one energy delivery element from the plurality of energy delivery elements and wherein the second area extends beyond the first area.

26. The system according to claim 21, wherein the system is configured to deliver the RF field and the PEF in a bipolar manner between two or more energy delivery elements from the plurality of the energy delivery elements.

27. The system according to claim 21, wherein the one or more generators include an RF generator configured to generate the RF signals; and wherein the one or more generators include a PEF generator configured to generate the PEF signals.

28. The system according to claim 27, wherein the PEF generator is configured to generate monophasic and biphasic PEF signals.

29. The system according to claim 27, wherein the PEF generator includes a plurality of voltage multiplier stages, and wherein each voltage multiplier stage includes at least one storage element and a plurality of semiconductor switches.

30. The system according to claim 29, wherein the at least one storage element is a capacitor, and wherein the PEF generator is configured to charge the capacitors included in the plurality of the voltage multiplier stages in a parallel configuration and to discharge the capacitors in a series configuration.

31. The system according to claim 29, wherein the PEF generator is configured to use each storage element included in the plurality of voltage multiplier stages to generate PEF signals.

32. The system according to claim 21, wherein the system is configured to deliver the RF field as a train of RF pulses, wherein the train of RF pulses has a duration of 0.01 s to 10 s.

33. The system according to claim 32, wherein the RF pulses within the train of RF pulses are delivered in a continuous manner.

34. The system according to claim 21, wherein the delivery of the PEF includes a delivery of at least one train of PEF pulses, and wherein a number of the PEF pulses delivered during the delivery of the PEF is from 1 to 10000 PEF pulses.

35. The system according to claim 21, wherein the delivery of the PEF includes a delivery of at least one PEF pulse, wherein the at least one PEF pulse has a pulse width of 2 ns to 10 ms.

36. The system according to claim 35, wherein the PEF pulse energy is from 0.01 J to 1 J, an energy delivered per PEF pulse per unit area is from 0.01 J/mm² to 1 J/mm², and/or wherein an energy delivered per PEF pulse per unit volume is from 0.01 J/mm³ to 1 J/mm³.

37. The system according to claim 21, wherein the plurality of energy delivery elements comprises from 4 to 200 energy delivery elements, and wherein each energy delivery element of the plurality of energy delivery elements is configured to deliver the RF field and the PEF.

38. The system according to claim 37, wherein the energy delivery elements are needle electrodes configured to penetrate the skin tissue, and wherein the needle electrodes are extendable, and wherein a distance from a tip of each energy delivery element to a surface of the applicator from which the energy delivery element is extended, is adjustable from 0.2 mm to 15 mm.

39. The system according to claim 37, wherein the energy delivery elements are surface electrodes configured not to penetrate the skin tissue.

40. A method of treating of a skin tissue comprising at least one of a hypodermis, dermis, or mucosal tissue, the method comprising:
providing one or more generators configured to generate radio frequency (RF) signals and pulsed electric field (PEF) signals;
providing at least one applicator including at least one energy delivery element, wherein the at least one energy delivery element is electrically coupled to the one or more generators;
applying an RF field and PEF from the one or more energy delivery elements to the skin tissue based on the RF signals and PEF signals from the one or more generators;
causing heating of the skin tissue via the RF field; and
causing an electroporation of cell membranes included in the skin tissue via the PEF;
wherein the application of the RF field results in destruction of cells and non-cellular tissue by heating in a first area in the skin tissue; and
wherein the application of the PEF results in destruction of cells by electroporation of cell membranes in a second area in the skin tissue.
